(19) United States
(12) Patent Application Publication
(10) Pub. No.: US 2004/0170656 A1
(43) Pub. Date: Sep. 2, 2004

(54) Powder refill with plastic material support and compact comprisinig said refill

(76) Inventor: Robert Petit, Savigny Sur Orge (FR)

Correspondence Address: YOUNG & THOMPSON, 745 SOUTH 23RD STREET 2ND FLOOR, ARLINGTON, VA, 22202

(21) Appl. No.: 10/479,989

(22) PCT Filed: Jun. 6, 2002
(86) PCT No.: PCT/FR02/01924

(30) Foreign Application Priority Data
Jun. 8, 2001 (FR) 0107528

(51) Int. Cl. A61K 7/00

(52) U.S. Cl. 424/401

(57) ABSTRACT

The invention concerns a cosmetic product refill (6) in the form of a compressed powder designed to be incorporated in a compact (1), comprising a plastic material support (7) and a block (8) of said compressed powder, said block (8) being secured to said support (7), and said support (7) comprising reversible or irreversible fixing means (70), co-operating with fixing means (10) of said housing (1), with predetermined dimensions, typically with a clasp (11) of overlap R less than 0.5 mm.

### FIELD OF THE INVENTION

The invention relates to the field of cosmetic product packaging, and more specifically to that of the powder compacts for facial care.

### STATE OF THE RELATED ART

As a general rule, compacts typically comprise:

a concave lid possibly equipped with an inner mirror,

a concave base, typically equipped with a grid or intermediate support of at least one cup designed to receive said cosmetic product,

a hinge, forming the link between said lid and said base, enabling the articulation of said lid and the opening of said compact,

a fastener or any locking and unlocking means of said lid with respect to said base, so as to open and close said compact, a fastener being typically a push button.

A large number of compact embodiments are known, said embodiments possibly applying to one or more of the constituent parts of the compact.

In this way, as regards the patents held by the applicant, it is possible to mention:

the patent FR 2 661 080 which discloses a makeup compact with a fastener of a compact size,

the patent FR 2 701 365 which discloses a convertible makeup compact,

the patent FR 2 725 958 which relates to a joined locking compact with assisted opening,

the patent FR 2 737 192 which discloses a compact with a pivoting lid equipped with a flexible tab hinge,

the patent FR 2 755 352 which discloses a compact with assisted opening,

the patent FR 2 755 353 which discloses a compact with an unlocking and pre-opening push button,

the patent FR 2 756 155 which discloses a makeup compact, of the type comprising a base, an intermediate plate and a lid equipped with an inner mirror.

Compacts comprising cups attached inside the compact are also known.

Problem Statement

In the field of cosmetic product packaging, there is a growing demand for packaging which illustrates or suggests transparency and compacts are no exception.

Contrary to what might be assumed, for this, it is not sufficient to select a transparent material to form said compact. Indeed, the drawback of transparency is the highlighting of parts of the compact which may not be very decorative, whether they are parts relating to the hinge, fastener, or the assembly of the cup or the refill with the compact itself.

Therefore, a major problem consists of rendering these parts as discreet and invisible as possible, particularly the assembly of the cup or the refill with the compact itself given that it is important that, visually, attention is not dispersed or drawn to anything other than the cosmetic product itself.

### DESCRIPTION OF THE INVENTION

According to the invention, the cosmetic product refill in the form of a compressed powder designed to be incorporated in a compact comprises a plastic material support and a block of said compressed powder, said block being secured to said support, and said support comprising reversible or irreversible fixing means to said compact, with predetermined dimensions, typically with a clasp of overlap less than 0.5 mm.

In the present invention, the term “refill” refers to the cup or support comprising the compressed powder, whether the cup is fixed reversibly or irreversibly to the base of the compact or a grid, the fixing being reversible when, in the compact, the cup is replaced once the powder has been used up.

In this way, the applicant observed that, using the means of the invention, it was possible to solve the problems posed and obtain refills that are both “discreet” by means of compact fixing means, and fixed to the compact in a reliable manner, by means of obtaining stable predetermined dimensions, in spite of having a plastic support.

### DESCRIPTION OF FIGURES

All the figures relate to the invention.

FIGS. 1a to 2e illustrate a first embodiment of a refill 7 and compact 1 according to the invention, FIGS. 1a to 1e relating to the compact 1 and FIGS. 2a to 2e relating to the refill 7, the compacts and refills being square in shape.

FIG. 1a is a perspective view of the closed compact 1.

FIG. 1b is a bottom view of the base 3 of the compact 1, the base having two compartments: the left compartment 30 is designed to receive two refills or cups 7 represented with dotted lines, while the right compartment 31 is designed to receive an application means, typically a powder puff.

FIG. 1c is a cross-section of the compact 1 perpendicular to the hinge 4.

FIG. 1d is an enlarged view of the front or fastener 5 of the compact 1 in FIG. 1c.

FIG. 1e is an enlarged view of the rear or hinge 4 of the compact 1 in FIG. 1c.

FIG. 2a is a top view of the support or cup 7.

FIG. 2b is a cross-section of the support 7 in FIG. 2a along the vertical axis shown in dotted lines in this figure.

FIG. 2c is a side view of the support 7 in FIG. 2a.

FIG. 2d is an enlarged view of FIG. 2c—part surrounded by dotted lines.

FIG. 2e is an enlarged view of FIG. 2b—part surrounded by dotted lines.

FIGS. 3a to 4 illustrate a second embodiment of a refill 7 and compact 1 according to the invention, FIGS. 3a to 3c relating to the compact 1 and FIG. 4 relating to the refill 7. This embodiment differs from the above particularly through the rectangular shape of the refill 7 and the compact 1.

FIGS. 3a, 3b and 3c are similar to FIGS. 1a, 1b and 1c, respectively.

FIG. 3d is an enlarged section along D-D of FIG. 3c.

FIG. 3e, similar to FIG. 3d, illustrates the overlap R of a clasp formed by the cooperation between the rim 100 of the fixing means 10 of the concave base 3 of the compact 1, and the clasp 701 of the tab 700 of the fixing means 70 of the support 7.

FIG. 4 is similar to FIG. 2a.

FIGS. 5a to 5c are sections in the vertical plane of the combined compression device 9 of the powder 80 and the support 7, so as to form a single-piece powder refill 6.

FIG. 5a represents the lower part of the device 9, typically fixed, comprising a die 90, forming a cavity 93 filled with powder 80.

FIG. 5b represents the upper part of the device 9, typically mobile in the axial direction, comprising a piston 92.

FIG. 5c illustrates the device 9 after compression, at least partial, of the powder, 80, to form the block of compressed powder 8 secured on the support 7.

### DETAILED DESCRIPTION OF INVENTION

According to the invention, said fixing means 10, 70 may be latching means, typically by means of the cooperation of male and female parts, one being comprised by said support 7, the other being comprised by said compact 1.

For this, said support 7 may comprise a rigid base 71 and/or a rigid lateral skirt 72 comprising said male or female part forming part of said fixing means. According to the invention, it is important that the fixing means are comprised by rigid parts belonging to the compact 1 and the support 7 in order to obtain fixing that is both reliable and compact.

Following the applicant's tests, it emerged that it was preferable to secure said support to the compact by means of latching or snap-on fastening, i.e. by a mechanical means, rather than by gluing given that, the compact being transparent, the visible gluing zone renders the bottom of the compact unattractive.

According to one alternative embodiment of the invention, said support 7 may consist of a rigid base 71. It may also consist of a rigid base and a flexible or rigid lateral skirt.

Said rigid base may also consist of a single rigid ring possibly surrounding a flexible or rigid central panel.

Typically, said support 7 forms a cup comprising a bottom of a base 71 and a lateral skirt 72.

However, particularly in view of the fact that said support 7 and said block 8 may form a single-piece part subject to combined compression, so that said block is secured to said support, virtually any type of support 7 may be suitable according to the invention, subject to the dual condition of being able to be secured to said powder block and in particular being able to be secured to the powder block by combined compressions, and of being able to be fixed to said compact.

To improve the cohesion between the powder block 8 and the support 7 if required, said support 7 may comprise raised or hollowed elements so as to form an additional means securing said block onto said support.

Said support is made of plastic material, typically polyolefin (PE, PP) or polyester (PET, PBT). It may have a thickness between 0.5 and 2 mm. This thickness is typically 1 mm+/−0.2 mm, at least for the part of the support comprising the fixing means 70, such that at least this part of the support may be considered as “rigid” in the sense of the present invention.

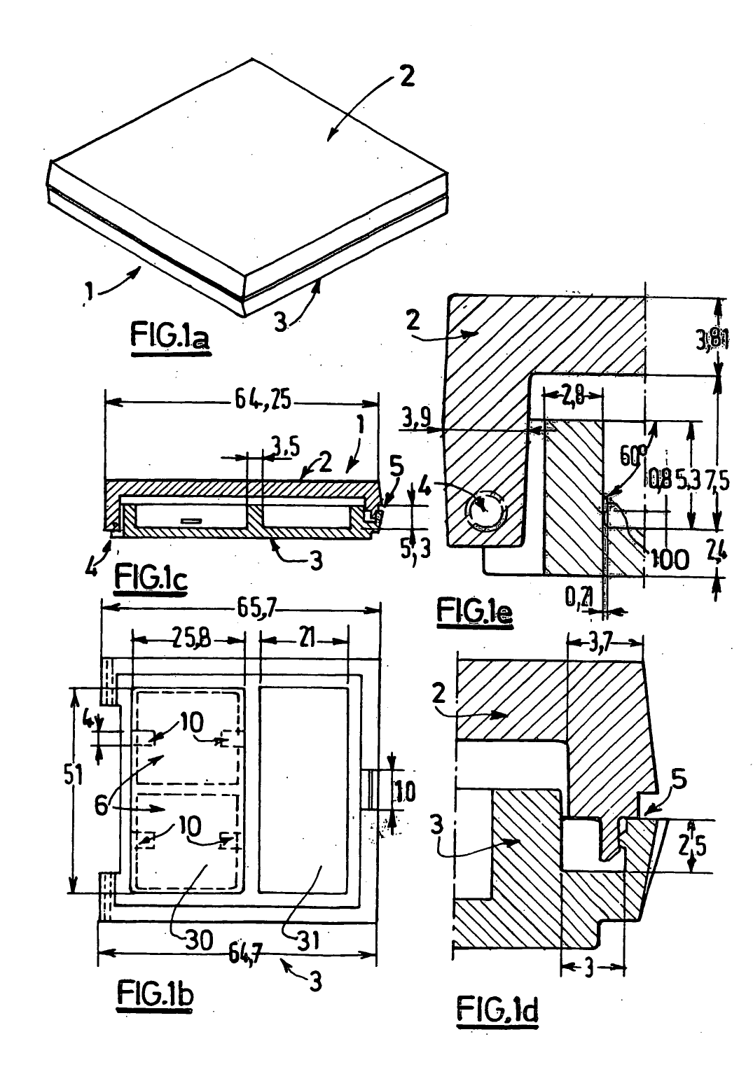

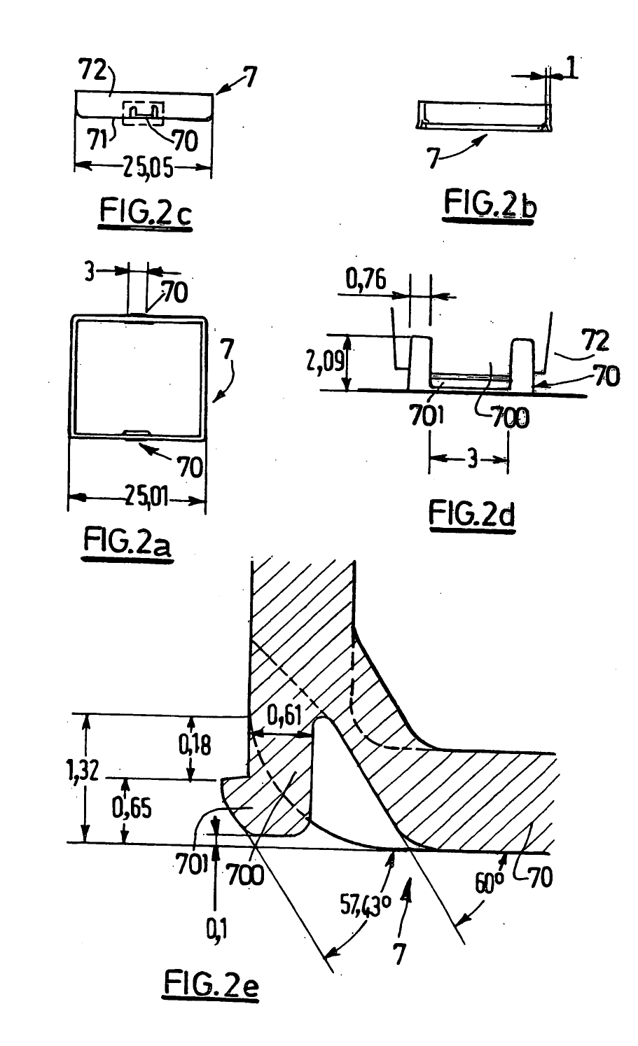

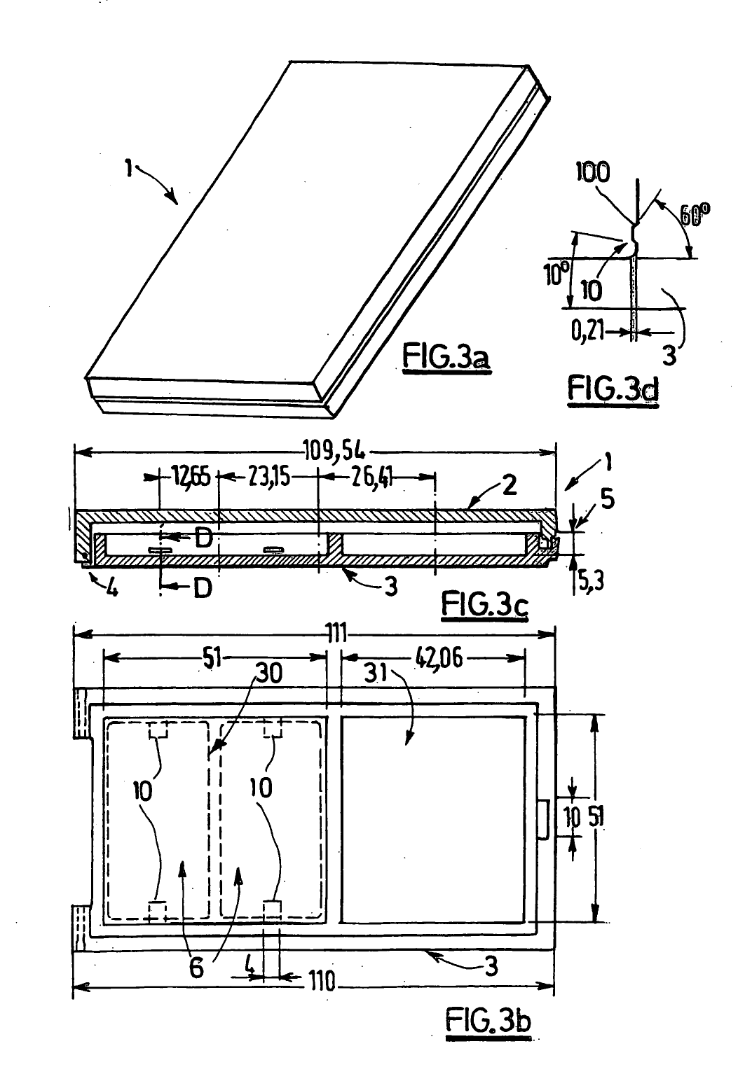

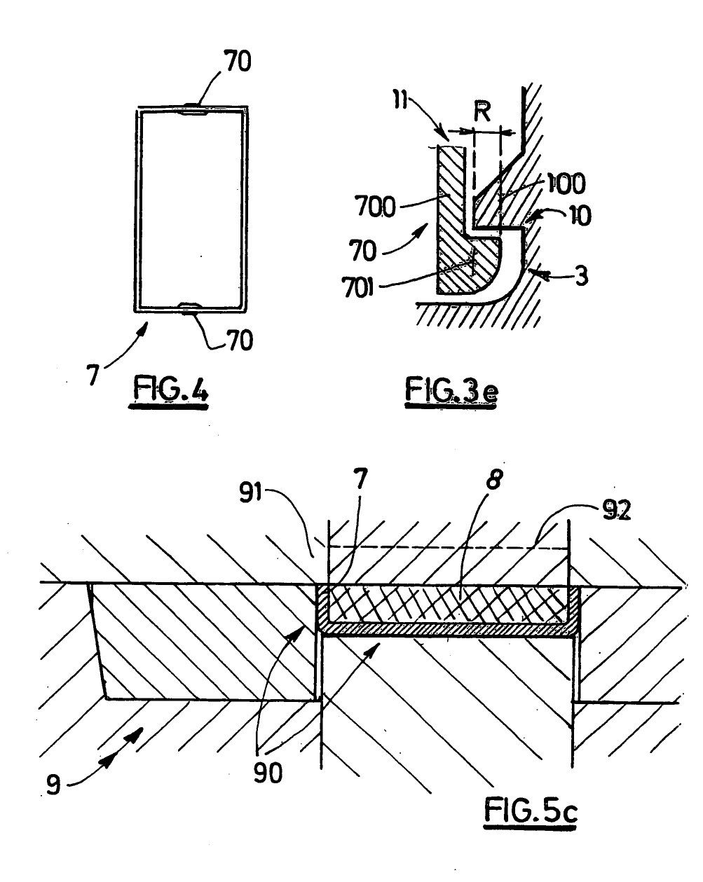

As illustrated in FIGS. 2a to 2e, said support may comprise, on its opposite faces, two flexible axial tabs 700 wherein the lower end forms a male part, typically in the form of a clasp 701, designed to cooperate with a female part, typically in the form of a recess or formed by a rim 100, located on an inner surface of said compact 1, the whole forming said fixing means. Such a rim is shown in FIGS. 1e and 3d.

According to the invention, the apparent density of said compressed powder block 8 may be between 0.8 and 2, and typically 1.5, and selected so as to obtain a compressed powder block both secured to said support 7 and showing a mechanical resistance and other properties in compliance with standard requirements for a powder block.

It is advantageous, according to the invention, for said refill and said compact to be fixed by means of a clasp with overlap R between 0.3 mm and 0.15 mm, and preferentially less than 0.2 mm, so that, visually, said technical means of the compact go virtually unnoticed. See FIG. 3e.

The invention is not limited to any refill shape. Indeed, the invention makes it possible to obtain refills which comprise in their horizontal plane any cross-section and/or which comprise a top surface of any shape or raised surface, as will be seen with the method to manufacture a refill according to the invention, which is described below.

The invention also relates to a method to manufacture a refill 6 according to the invention.

In this method:

a) said powder 80 and said support 7 are supplied, said support being fitted to said compact 1,

b) a powder compression tool 9 is used, comprising a compression die 90 of a shape suited to said support 7, comprising an assembly of several parts, including a tubular compression part 91, forming an open cavity 93, and an axial compression piston 92 capable of moving in said tubular compression part 91 or in said open cavity, and said support 7 is placed in said compression die 90, typically at the bottom of said open cavity, so that said support is substantially immobilised in said cavity,

c) a predetermined quantity of said powder 80 is filled into said cavity 93,

d) said predetermined quantity of powder is compressed using said compression piston 92, by axial compression, to form a block of powder of volume V secured to said support, thus creating an axial compression strain,

e) said axial compression strain is released such that the internal strains of said powder block are dissipated axially and said block has reached its definitive volume V′ without strain where V′>V, such that said refill 6, before being removed from said tool, forms a single-piece part subject to combined compression of a stabilised geometry consisting of said powder block 8 secured to said support 7 and that, in this way, said refill 6 has a dimensional stability enabling its fixation to said compact 1.

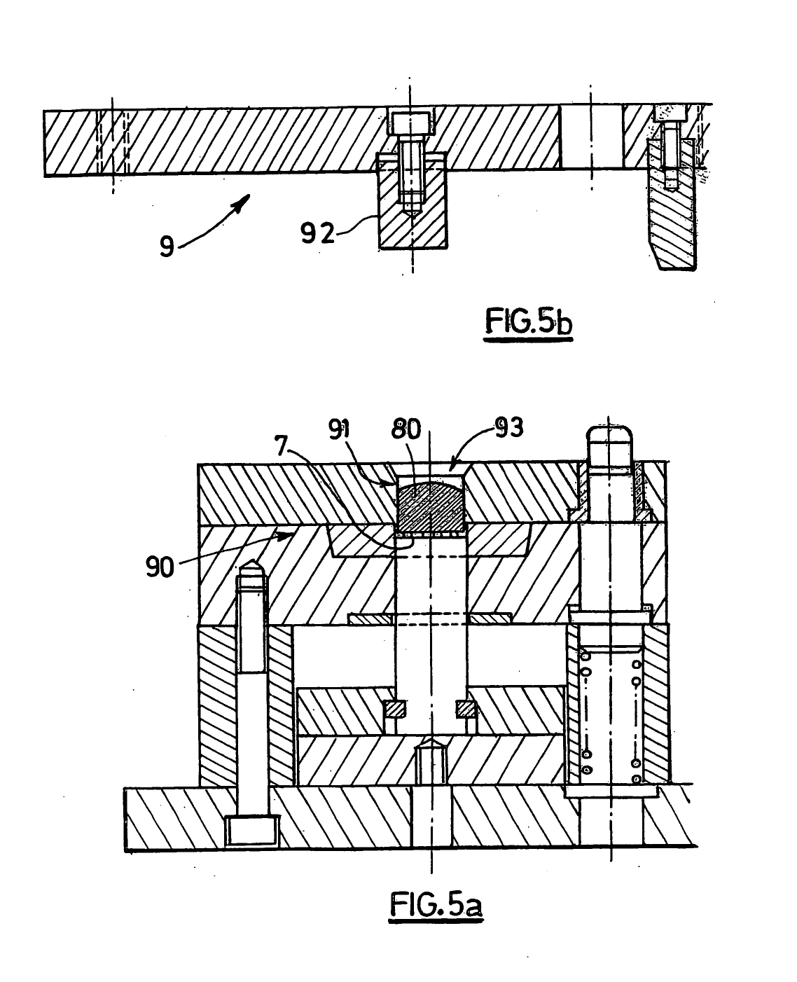

FIGS. 5a to 5c illustrate this method.

As can be seen in these figures, the support 7 forms a cup wherein the outer surface is completely surrounded by a compression die 90, typically formed of several parts. Similarly, the upper rim of the cup is masked by the inner wall of the tubular part 91, such that the piston 92 could slide in said cup if applicable. Therefore, such a support or cup 7 is completely immobilised during said combined compression.

As observed by the applicant, it is important that the refill 6 formed by combined compression remains secured to the die 90 while strains remain with said refill.

In practice, the compression strains are allowed to dissipate axially, by slight expansion of the powder block 8 when the piston 92 is removed, while the refill is still held in the die 90.

Using these means, the applicant observed that it was possible to obtain plastic supports or cups 7 of great dimensional stability and precision and with compact fixing means to the compact, so as to be able to fix said support 7 to said compact 1 in a reliable and discreet manner.

The invention also relates to the compact 1 comprising a lid 2 and a concave base 3 secured by a hinge 4 and a fastener 5, and possibly a grid, and at least one refill 6 according to the invention, said refill 6 being secured to said base 3 or to said grid, typically by latching. The compacts represented in the figures do not comprise a grid, the support 7 being directly fixed on the inner wall of the concave base 3 of the compact 1.

According to the invention, said lid 2, and/or said base 3, and said grid if applicable, are made of transparent plastic materials.

With respect to said support 7, it may:

either be coloured throughout,

or have a colour selected according to the colour of said powder block 8, and is typically a similar colour or a complementary colour,

or possibly be made of a transparent material, like the compact itself.

It is advantageous, in the compact 1 according to the invention, for said hinge 4 and said fastener 5 to be located in the thickness of the material forming said lid 2 and said base 3 and typically only comprise transparent materials.

These transparent materials may typically be selected from the following polymers: PS (polystyrene), PET (polyester), PC (polycarbonate), MPMA (methyl polymethacrylate), MBS (methacrylate butadiene styrene copolymer), SAN (styrene acrylonitrile copolymer).

### EXAMPLES OF EMBODIMENTS

FIGS. 1a to 2e represent a first example of an embodiment of a square compact 1 comprising two refills 8 comprising a support 7 in the form of a square cup.

FIGS. 3a to 3e represent a second example of an embodiment of a rectangular compact 1 comprising two refills 8 comprising a support 7 in the form of a rectangular cup.

FIGS. 5a to 5c illustrate the combined compression device used to manufacture the refills 8 manufactured in these two examples of embodiments.

The compacts 1—lid 2, concave base 3 and hinge 4 and fastener 5—were manufactured by moulding SAN, a transparent plastic material. The supports 7 were manufactured in the form of cups by moulding PBT or PP.

Advantages of the Invention

The invention offers several advantages:

firstly, it makes it possible to solve the problems posed,

secondly, the invention opens the way for a large number of alternative embodiments or forms of compacts, the invention not being limited to the examples of embodiments alone. In particular, although only irreversible fixing means have been illustrated in the figures, it is possible according to the invention to fix said refill 6 in a reversible manner, particularly by showing once the powder has been used up the possibility to “unlatch” said support and thus replace said refill 6,

finally, otherwise, it makes it possible to obtain compacts that are substantially more economical than standard compacts. Indeed, the manufacture of a refill is carried out in a single step, possibly using economical materials, during which the powder is compressed and the powder block formed is fixed on its support simultaneously.

List of References

compact 1

fixing means 10

rim 100

overlap clasp 11

lid 2

concave base 3

refill housing 30

powder puff housing 31

hinge 4

fastener 5

powder refill 6

support 7

fixing means 70

tabs 700

clasp 701

base 71

skirt 72

compressed powder block 8

powder 80

compression tool 9

compression die or casing 90

tubular part 91

piston 92

cavity 93

1. Cosmetic product refill (6) in the form of a compressed powder designed to be incorporated in a compact (1) comprising a support (7) and a block (8) of said compressed powder, said block (8) being secured to said support (7) and being formed by compressing a powder in said refill, said block and said support thus forming a single-piece part subject to combined compression, and said support (7) comprising reversible or irreversible fixing means (70) co-operating with fixing means (10) of said compact (1), by latching male and female parts, characterised in that:
a) said support is made of plastic material,
b) said single-piece part is a single-piece part with stabilised geometry, such that said single-piece part has stable predetermined dimensions,
c) said fixing means (70) and (10) form a clasp (11) of overlap R less than 0.5 mm, said support (7) comprising a rigid base (71) and/or a rigid lateral skirt (72) comprising said male or female part forming part of said fixing means.

2. Refill according to claim 1 wherein said support consists of a rigid base (71).

3. Refill according to claim 1 wherein said support consists of a rigid base and a flexible or rigid lateral skirt.

4. Refill according to claim 2 wherein said rigid base consists of a rigid ring possibly surrounding a flexible or rigid central panel.

5. Refill according to any of claims 1 to 4 wherein said support (7) comprises raised or hollowed elements so as to form an additional means securing said block onto said support.

6. Refill according to any of claims 1 to 5 wherein said support is made of polyolefin or polyester.

7. Refill according to any of claims 1 to 6 wherein said support is between 0.5 and 2 mm thick.

8. Refill according to any of claims 1 to 7 wherein said support comprises two flexible axial tabs (700) wherein the lower end forms a male part, typically in the form of a clasp (701), designed to cooperate with a female part, typically in the form of a recess or formed by a rim (100), located on an inner surface of said compact (1), the whole forming said fixing means.

9. Refill according to any of claims 1 to 8 wherein the apparent density of said compressed powder block (8) is between 0.8 and 2.

10. Refill according to any of claims 1 to 9 wherein the overlap R of said overlap clasp is between 0.3 mm and 0.15 mm, and preferentially less than 0.2 mm.

11. Refill according to any of claims 1 to 10 comprising in its horizontal plane any cross-section and/or comprising a top surface of any shape or raised surface.

12. Method to manufacture a refill (6) according to any of claims 1 to 11 wherein:
a) said powder (80) and said support (7) are supplied, said support being fitted to said compact (1),
b) a powder compression tool (9) is used, comprising a compression die (90) of a shape suited to said support (7), comprising an assembly of several parts, including a tubular compression part (91), forming an open cavity (93), and an axial compression piston (92) capable of moving in said tubular compression part (91) or in said open cavity, and said support (7) is placed in said compression die (90), typically at the bottom of said open cavity, so that said support is substantially immobilised in said cavity,
c) a predetermined quantity of said powder (80) is filled into said cavity (93),
d) said predetermined quantity of powder is compressed using said compression piston (92), by axial compression, to form a block of powder of volume V secured to said support, thus creating an axial compression strain,
e) said axial compression strain is released such that the internal strains of said powder block are dissipated axially and said block has reached its definitive volume V′ without strain where V′>V, such that said refill (6), before being removed from said tool, forms a single-piece part subject to combined compression of a stabilised geometry consisting of said powder block (8) secured to said support (7) and that, in this way, said refill (6) has a dimensional stability enabling its fixation to said compact (1).

13. Compact (1) comprising a lid (2) and a concave base (3) secured by a hinge (4) and a fastener (5), and possibly a grid, and at least one refill (6) according to the any of claims 1 to 11, said refill (6) being secured to said base (3) or to said grid, typically by latching.

14. Compact according to claim 13 wherein said lid (2), and/or said base (3), and said grid if applicable, are made of transparent plastic materials.

15. Compact according to any of claims 13 to 14 wherein the support (7) of said refill (6) is coloured throughout.

16. Compact according to claim 15 wherein the colour of said support (7) selected according to the colour of said powder block (8), and is typically a similar colour or a complementary colour.

17. Compact according to any of claims 13 to 16 wherein the support 7 of said refill (6) is made of a transparent material.

18. Compact according to any of claims 13 to 17 wherein said hinge (4) and said fastener (5) are located in the thickness of the material forming said lid (2) and said base (3) and typically only comprise transparent materials.
